(19) United States
(12) Patent Application Publication
(10) Pub. No.: US 2016/0175826 A1
(43) Pub. Date: Jun. 23, 2016

(54) CATALYST AND RELATED METHODS

(71) Applicant: Albert R. Duoibes, East Grand Rapids, MI (US)

(72) Inventor: Albert R. Duoibes, East Grand Rapids, MI (US)

(21) Appl. No.: 15/053,625

(22) Filed: Feb. 25, 2016

Related U.S. Application Data
(63) Continuation-in-part of application No. PCT/US14/52018, filed on Aug. 21, 2014.

(51) Int. Cl. B01J 31/00; C07K 2/00; C07K 1/107; C07H 3/02; C07C 319/14; C07C 227/12; C07C 277/08; C07D 233/64; C07D 207/16; C07D 209/20; B01J 31/02; C07H 15/12

(52) U.S. Cl. CPC B01J 31/00; B01J 31/003; B01J 31/0237; C07K 2/00; C07K 1/107; C07H 3/02; C07H 15/12; C07C 227/12; C07C 277/08; C07D 233/64; C07D 207/16; C07D 209/20; C07C 319/14

(57) ABSTRACT

Fossilized organic matter (FOM) is used as a catalyst for reactions including but not limited to nitrogen fixation, glycosylation, amino acid/protein synthesis, glycolysis, carbon fixation.

### CLAIM OF PRIORITY

This application is a continuation-in-part of and claims priority to PCT application Serial No. PCT/US2014/052018 filed, Aug. 21, 2014, entitled CATALYST AND RELATED METHODS, which in turn claims priority to provisional patent application Ser. No. 61/870,123, filed Aug. 26, 2013, entitled CATALYST AND RELATED METHODS.

### FIELD AND BACKGROUND OF THE INVENTION

The present invention relates to catalysts, methods of making them, and their uses.

### SUMMARY OF THE INVENTION

In the present invention, fossilized organic matter (FOM) is used as a catalyst for reactions including but not limited to nitrogen fixation, glycosylation, amino acid/protein synthesis, glycolysis, carbon fixation.

### BRIEF DESCRIPTION OF THE DRAWINGS

FIG. 1 illustrates the process of extraction of fossilized organic matter (FOM) from fossilized soil (FS); and

FIG. 2 outlines a process for catalyzing reactions using FOM.

### BRIEF DESCRIPTION OF THE PREFERRED EMBODIMENTS

### Introduction

The term catalyst is used herein to include material for which a very small amount causes desired reactions with a much larger amount of a reactant or reactants. The material in this case, fossilized organic matter, comprises numerous different components. The precise identity of some of the components is not known. The extent to which each component may participate in any given reaction is not known. The exact manner in which FOM or its individual components acts as a catalyst as defined herein is not known. The extent to which the individual components may react with each other in the reaction is not known. The extent to which the FOM or any of its components is creating any other catalyst in the reaction process is not known. The extent to which the FOM or any of its individual components is consumed or eventually spent in the reaction process is not known. It is not recovered as an intact entity upon completion of the reactions described herein. What is known, is that a very small quantity of FOM can be used to create a reaction involving a much larger quantity of reactant or reactants, which produces a desired result.

The term “fossilized organic matter” (FOM) as used herein refers to organic matter derived from fossilized soil. The fossilized organic matter is derived from the fossilized soil by a process of solvent extraction, with water being the preferred solvent. The extracted fossilized organic matter is optionally sterilized, depending on the intended uses. Iron contained in the FOM is preferably complexed with phosphate, in the form of iron (III) phosphate, thereby rendering it insoluble to slightly soluble in water, depending on pH. The iron thus complexed may optionally be removed from the fossilized organic matter prior to using it as a catalyst.

### Fossilized Soil Selection

The term “fossilized soil” refers to soil containing plant organic material in which the cell walls and fibrous material making up plant material has been removed, leaving behind cellular cytosol materials, including without limitation, crystalline (shales) organic mineral material, amino acids, saccharides, enzymes, cellular salt complexes and other as yet unidentified components that can be solubilized or colloidalized in water, and thereby water extracted from the fossilized soil. The fossilized soil preferably has a significant lignite and Leonardite component in it. The organic material contained in the fossilized soil can be said to have been fossilized, that is to constitute fossilized organic matter (FOM).

The fossilized organic matter is fossilized plant material, including plant breakdown products and/or plant breakdown byproducts. The “organic matter” may include those decomposition products which may be formed when plant material is mixed with soil to promote plant matter breakdown when left in such soil over enough time to form rich soils. The plant material in such soils may have become “fossilized” over the years under natural compressive “forces” or volcanic or tectonic events, and may have the beneficial properties and uses of preserving plant material in “fossilized” form.

Eventually, accumulated plant materials disintegrate and are broken down by the natural biological processes associated with earthen microflora and microfauna found in soils in combination with the other forces described herein. Accordingly, some soils may be rich in plant materials while others may not be so enriched. For example, a surface layer of desert sand (e.g., sand from the hottest, most arid and least hospitable part of the Sahara Desert at its most severe waterless spot; or desert sand from an equally inhospitable location elsewhere) would likely be considered soil poorly enriched by plant material.

The preferred soils are those soils that are rich in (or richer in) the quantity, age and/or type of plant material, plant breakdown products and/or plant breakdown byproducts present therein, referred to herein as fossilized organic material (FOM). Without being bound by theory, the supply of plant material present in the soil may continually (or may continuously) be undergoing breakdown in the presence of the microbial life (e.g., by way of one or more of bacteria, fungi, yeast, mold etc.) living (or present) in the soils under the conditions the soil was exposed to over the course of its geographic history. It is also possible that the breakdown of plant material into plant breakdown products and byproducts may be enhanced under certain soil conditions including, but not limited to, water content, soil drainage, longitude and latitude of soil location, soil density, sand content, salt content, salt type(s), mineral content, elements present, metals presents, other soil constituents present or absent, soil pH, clay content, soil type, soil porosity, age of soil, depth of soil sample, temperature, pressure, sunlight, duration of day, climate zone, extent of aerobic or anaerobic surroundings, the quantity and identity of microbial life active in the soil, nutrients present, nutrient quantity, any changes in one or more of the above over time, and/or some other parameters including any combination or sub-combination of any of the above.

The “organic matter” is itself “fossilized organic material.” Such “fossilized organic material” in question may be obtained from fossilized soil (FS) samples containing breakdown products and breakdown byproducts of disintegrated plant material within the soil. Preferably, the fossilized organic material is plant material. While the fossilized organic material may possibly contain some non-plant organic material at some point in time, it is preferably degraded and preferably constitutes less than 10% by volume of the organic material present.

Exemplary fossilized soils are found in the St. Bernard Delta Basin and the LaFourche Delta basins formed by the Mississippi River from about 1800 to about 4600 years ago (Mineralogic Study of Sediments from Nearshore Cat Island, Miss., pp. 14 and 24, Laura Belle Barnhart, a thesis submitted to the Faculty of Mississippi State University, 2003). Another suitable source of FS is the Vicksburg Group geologic formation, and especially the Bucktoona Clay unit (Soil Survey of Smith County Mississippi, pp. 11 and 150, Ralph M. Thornton, 2001). Layers of FS can typically be found at 10 to 100 feet of depth.

In one more specific embodiment, the (FS) can be found are centrally located at Mt. Olive, Miss. Locations that may be used for (FS) collection are situated on two areas having approximately 20+ acres in their central portion. The first of these particular areas where the fossilized soil (FS) may be collected is at longitude 89° 39′13″W and latitude 31° 45′24″N or within a radius of about 1,000 acres in any direction from the parcel's center. The second particular area where the fossilized soil (FS) may be collected is at longitude 89° 39′13″W and latitude 31° 4524″N or within a radius of about 1,000 acres in any direction from the area center.

### Fossilized Organic Matter Plant Type

Optionally, the fossilized organic material (from soils of interest) may include: grasses, weeds, any leaf, any flower, any seed, any seed pod, any outer plant or fruit layer, bark, juice, pulp, outer scales, needles, thorns, petals, flower stamen's, pollen, stems, branches, wood, limbs, connective tissue, sap, syrup, roots, or other plant parts, etc., irrespective of whether the plant is alive and growing in open-air, underwater, underground or elsewhere so long as such fossilized parts are of a species or of a species relying on photosynthesis or requiring sunshine to grow, which would generally be a vegetable, a grass, a plant, a fruit, a root, a seed, a bean, a string bean, corn, corn husk, husk, a leaf, a branch, a bark, an under-layer or internal part of any plant thereof, and/or any combination thereof.

Extraction of Fossilized Organic Matter from Fossilized Soil

With respect to isolating, separating or extracting “fossilized organic matter” from “fossilized soils,” the extraction solvent is a polar solvent. Examples of such solvents include, but are not limited to, distilled water, de-ionized water, tap water, water of any quality sufficient to conduct sufficient extraction without materially interfering with commensurate operational efficiency. Water locally collected from the same site or near the site where the soil sample is collected (or is intended to be collected from) may also be used. The extraction is preferably conducted in water at a slightly acidic pH of less than about 7, preferably from about 4 to about 7, more preferably, at a pH of about 5-6.

The water may be mixed with a co-solvent. The co-solvent may be a water-miscible co-solvent better suited to extracting the sought soil constituent(s) of interest. Co-solvents can include organic solvents such as, but not limited to, alcohols (methanol, ethanol, and the like), dimethylsulfoxide (DMSO), N,N-dimethylformamide (DMF), acetonitrile, tetrahydrofuran (THF), p-dioxane, and the like.

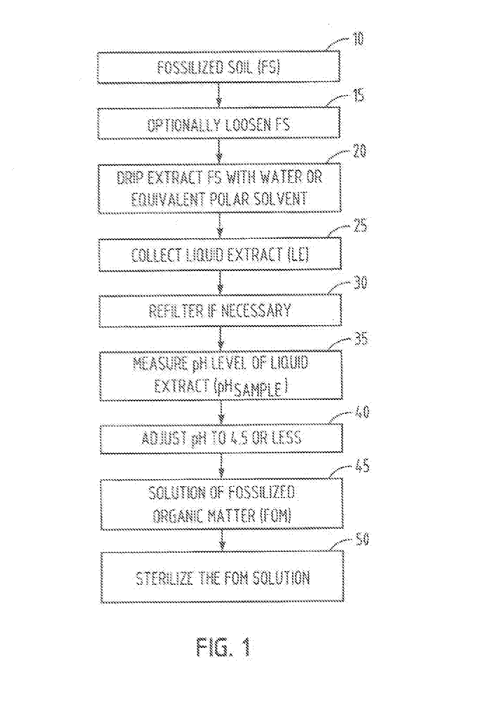

FIG. 1 illustrates the process of extraction of fossilized organic material from the fossilized soil. The process begins with a fossilized soil at 10. Optionally, the fossilized soil may be loosened to facilitate fluid flow through the soil rather than over it (15).

A drip extraction process is carried out using an appropriate solvent (e.g., water, distilled water, ground water, ionized water, sterile water, etc.) (20). The extraction solvent is poured on top of the fossilized soil in a batch process, or is mixed with the fossilized soil in a continuous process, and the liquid is passed through a sized filter after passing through or being mixed with the fossilized soil. The filter is sized to allow the passage of colloidal material, as well as the soluble material, as such colloidal material may contain desirable ingredients. To the extent necessary, the liquid is refiltered to eliminate undissolved and non-colloidal solids. The liquid extract (LE) is collected as noted at 25, and its pH level is measured (pHₛₐₘₚₗₑ) as noted at 30. The liquid extract contains the FOM, and indeed is a solution/colloidal suspension of the FOM.

Any suitable form of extraction other than drip extraction may be used. Where an equivalent of extraction is available, then such equivalent may be used if appropriate as would be recognized by one of ordinary skill in the art provided with the benefit of the disclosure of this application.

The time interval for measuring the pH level of the drip extracted liquid extract (LE) may be accomplished by continuous monitoring of the drip extraction collected liquid extract (LE)—for example by use of a pH meter connected to a pH probe ultimately connected to a monitoring computer or may be monitored manually or my use of pH paper and color change or by pH indicator and color change or an equivalent of the same. The pH monitoring could be at certain intervals of time rather than continuous monitoring as could be accomplished by a computer. The purpose of monitoring the pH of the liquid extract is to confirm that its pH has fallen to an acidic level, namely less than about 7. This is an indication that the proper fossilized organic material is being extracted.

Once the extraction is complete, it is important to take a final pH measurement. If the final pH is above 4.5, the pH is preferably adjusted to 4.5 or less, since the reaction of the fossilized organic matter (FOM) with saccharides is preferably conducted in solution/colloidal suspension/slurry at a pH of 4.5 or less.

### Optional Sterilization

Sterilization of the fossilized organic matter catalyst may or may not be desirable. The liquid extract may be sterilized to yield a sterilized (LE) denoted herein as (SLE), at 50.

Preferably, sterilization may be accomplished by heating, filtration, UV light, pasteurization or by all methods other than by pasteurization. Sterilization may not be possible by use of a 0.22 micron filter because it may be that the LE contains ingredients that would not pass through a 0.22 micron filter while still keeping the desirable particulate matter within the (LE). If necessary, it may be desirable to use a 0.22 micron filter, and then to separately collect any solids and heat those solids to a sufficient temperature and for a time to guarantee sterility of the solids so collected via use of a 0.22 micron or other suitable filter or filters. Thereafter, once the solids are chemically or heat treated to achieve sterility of the solids so as to render any pathogenic or other bacterial, microbial, viral, toxic or other contents harmless, such solids could be recombined with the filter sterilized (LE) using a 0.22 micron filter. In such manner, any heat labile dissolved contents of the LE could be spared heat or chemical degradation by heat or chemical sterilization reserved just for solids collected.

By such sterilization process, both dissolved heat labile or chemical labile (LE) constituents could be sterilized via filtration with a 0.22 micron filtration method. And solids that are not heat or not chemical treatment labile could be recombined with the sterilized (LE) obtained via 0.22 micron filtration. In such manner, those desirable solids could be preserved and sterilized if possible and those desirable heat labile or chemical labile dissolved (LE) constituents could be preserved for use in the form of a more potent or desirable (SLE). The relevant point being that whatever sterilization techniques are suitable may be used if so desired.

Of course, if the sterilization procedure is too expensive compared to another method or is faster compared to another equally suitable method, then one may opt for the best, cheapest, most efficient of methods or some combination of the above to reduce cost and increase productivity. In effect, one may use a less elegant sterilization method if such method accomplished the sought objective. Ultimately, heat sterilization may be utilized so long as conducted under conditions sufficient to sterilize without the excessive loss of stability. The resulting sterilized (LE) is denoted as (SLE).

Also, sterilization may be conducted at (50) or at any other point along the processes of FIG. 1, 2, or 3 so long as the desired product is obtained at the end. Where sterilization is unnecessary, the process at (50) could be skipped altogether.

### Complexing and Optional Removal of Iron

Iron is preferably complexed with phosphate, in the form of Iron (I) phosphate, thereby rendering it insoluble to slightly soluble in water, depending on pH. The iron thus complexed may optionally be removed from the fossilized organic matter prior to reacting it with saccharides. The FOM solution as extracted is placed in a first precipitator and a soluble phosphate or other precipitant is added to precipitate the iron. When substantially all of the iron has precipitated out of the solution and settled, the supernatant is separated from the iron precipitate, as indicated at. While most of the iron will thus be removed, some complexed iron may still remain in the FOM solution.

### Exemplary Catalytic Reactions

Fossilized organic matter acts as a catalyst for reactants including, but not limited to, carbohydrates, proteins, and/or amino acids. Exemplary reactions which are catalyzed by catalytic quantities of the fossilized organic matter include:
Nitrogen fixation, in which nitrogen is withdrawn from the air or other source of nitrogen to react with the carbohydrates to form amino acids;
Polymerization of the amino acids to form proteins;
Glycolysis, which includes decomposition of carbohydrates, including polymeric or oligomeric sugars into monosaccharides and carboxyl groups, and the rearrangement thereof,
Glycosylation, in which a sugar molecule is added to the nitrogen atom in a protein or amino acid;
Carbon fixation, in which carbon from the air or other source of carbon dioxide is incorporated into carbohydrates.

These reactions are preferably conducted in aqueous solutions/suspensions/slurries, under conditions similar to those exemplified below for catalyzing such reactions involving saccharides. A very small amount of catalyst, referred to as a catalytic amount, is used relative to the reactant or reactant being catalyzed. A preferred range, for example, taken as parts catalyst (as solids) to parts reactant(s) (as solids) would be from about 1:50 to about 1:2500, more preferably 1:100 to 1:2000, more preferably 1:200-1:1500. The “parts” referred to are parts as solids, even though reaction is conducted in solution/suspension/slurry.

Catalyzing Saccharide Based Reactions with Fossilized Organic Matter

The forgoing reactions can be effected, for example, by reacting a catalytic amount of fossilized organic matter with saccharides. The FOM may be sterilized or unsterilized, as desired by the circumstances. Similarly, it may be iron free or it may include complexed iron.

The process is carried out in solution/suspension/slurry at a pH of 4.5 or less, using from about 1:50 to about 1:2500, more preferably 1:100 to 1:2000, more preferably 1:200-1:1500 parts catalyst to parts saccharides. The reaction is preferably conducted at room temperature, preferably in a temperature range of from about 15° C. to about 30° C. The reaction is conducted in air or some other nitrogen and CO₂ containing environment. Optionally, different forms of light may be employed to enhance particular reactions which may be desired. Similarly, other reactants could be included in the reaction mixture to obtain desired end products.

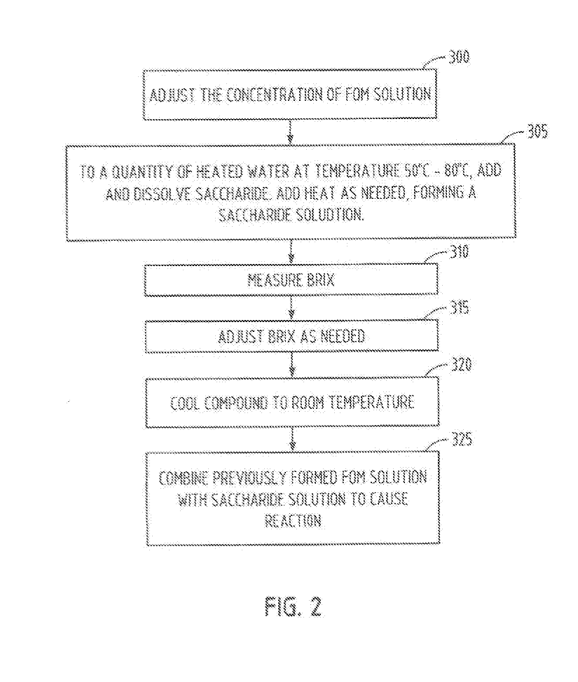

The volume of liquid in the liquid extract containing the fossilized organic matter is adjusted so that the concentration of FOM is from approximately 10 to about 80%, preferably about 20 to about 40%, and most preferably about 25 to about 35%. (FIG. 2 at 300) Separately, a quantity of water is heated to a temperature of about 50-80° C. (305). Then a saccharide material is added to the heated water (Qw) at a ratio of one gram of saccharide for every 2 milliliters of water (305). The saccharide is preferably substantially dissolved. Heat and an emulsifying agent may be added to aid in dissolution to form the saccharide solution (SS) (300). The Brix level is measured (310). It is preferably between about 20 and 80, more preferably about 35-67, most preferably about 50-55. If it is not, an amount of water or saccharide should be added as needed (315) until the Brix level is within this approximate range. Once the Brix level is attained, the saccharide solution (SS) solution should be cooled to room temperature (320).

The previously formed FOM solution/colloidal suspension (300) is combined with the SS, in air or some other nitrogen and carbon dioxide environment, to initiate a reaction. (325) Typically in a commercial batch, substantial reaction products are created in about 24 hours.

### Continuous Flow Process

The catalytic reactions of the preferred embodiments may also be conducted in a continuous flow process. The FOM catalyst is impregnated onto a high surface area solid catalyst support such as carbon, alumina, zeolites, silica, or polymeric resins. Resin supports include polystyrene, polystyrene-divinylbenzene, PVPDC, BEMP, polysiloxane and others. Impregnation or loading is achieved by suspension of or otherwise soaking of the solid support in a liquid extract solution of FOM at an FOM concentration of from approximately 10 to about 80%, preferably about 20 to about 40%, and most preferably about 25 to about 35%. Preferably, from about 10-25% FOM is loaded onto the support. After a sufficient time to achieve impregnation, the impregnated support is gently dried and prepared for use, typically in reaction columns or horizontal reactor tubes. The FOM solution is at a basic pH, usually about pH 8, during impregnation or loading.

The reactants, such as a saccharide solution (SS) with a Brix of preferably between about 20 and 80, more preferably about 35-67, most preferably about 50-55 is passed through the column. The saccharide solution is preferably buffered to a mildly acidic pH, preferably about 4 to 6. Mild heat and pressure are preferably used. The reaction is done in an air, nitrogen or carbon dioxide environment, as discussed above.

Example of an FOM Catalyzed Reaction with Saccharide

In this example, one milliliter of FOM solution at about 30% was reacted with a liter of sucrose solution at 54 Brix, or in other words about 54%. The reaction was conducted in air, in accordance with the procedure described above. The reaction products were analyzed for amino acids/proteins, sugars and amino sugars, carbohydrates, ash, fat and moisture. Proteins formed were generally but not specifically identified. Instead, any proteins formed were hydrolyzed into their amino acids for identification. The results are set forth in Tables 1-3 below:

TABLE 1
Sugars, Amino Sugars And Amino Acids With Carbon Content Indicated
Original Solution: 100 mL Sucrose in H2O @ 54% M/V
Total grams elemental Carbon in 54 grams C12H22O11: 22.74

| Final Product | | | | | | |
|---|---|---|---|---|---|---|
| Analyte | Result | Unit | Total g | Molecular Mass | C per Molecule | Carbon (g) |
| Mannose | 1.973 | % wt/v | 1.973 | 180.16 | 6 | 0.7892 |
| Xylose | 5.0472 | % wt/v | 5.0472 | 150.13 | 5 | 2.0188 |
| Arabinose | 1.2444 | % wt/v | 1.2444 | 150.13 | 5 | 0.4977 |
| Galactose | 4.0199 | % wt/v | 4.0199 | 180.16 | 6 | 1.6079 |
| Fructose | 0.0005 | % wt/v | 0.0005 | 164.16 | 6 | 0.0002 |
| Glucose | 4.9859 | % wt/v | 4.9859 | 180.16 | 6 | 1.9942 |
| N-Acetylneuraminic acid | 4.7759 | % wt/v | 4.7759 | 309.27 | 11 | 2.0401 |
| N-Acetylgalactosamine | 1.8467 | % wt/v | 1.8467 | 221.21 | 8 | 0.8021 |
| N-Acetylglucosamine | 10.8142 | % wt/v | 10.8142 | 221.21 | 8 | 4.6970 |
| Ala | 0.0055 | % wt/v | 0.0055 | 89.09 | 3 | 0.0022 |
| Arg | 0.0055 | % wt/v | 0.0055 | 174.2 | 6 | 0.0023 |
| Asx | 0.0055 | % wt/v | 0.0055 | 132.12 | 4 | 0.0020 |
| Cys | 0.2844 | % wt/v | 0.2844 | 121.16 | 3 | 0.0846 |
| Glx | 2.3521 | % wt/v | 2.3521 | 146.14 | 6 | 1.1598 |
| Gly | 0.0055 | % wt/v | 0.0055 | 75.07 | 2 | 0.0018 |
| His | 0.0055 | % wt/v | 0.0055 | 155.15 | 6 | 0.0026 |
| Ile | 0.0656 | % wt/v | 0.0656 | 131.17 | 6 | 0.0360 |
| Leu | 7.3243 | % wt/v | 7.3243 | 131.17 | 6 | 4.0237 |
| Lys | 0.0055 | % wt/v | 0.0055 | 146.19 | 6 | 0.0027 |
| Met | 0.0055 | % wt/v | 0.0055 | 149.21 | 5 | 0.0022 |
| Phe | 0.5853 | % wt/v | 0.5853 | 165.19 | 9 | 0.3830 |
| Pro | 0.0055 | % wt/v | 0.0055 | 115.13 | 5 | 0.0029 |
| Sar | 0.0055 | % wt/v | 0.0055 | 105.09 | 3 | 0.0019 |
| Thr | 0.7494 | % wt/v | 0.7494 | 119.12 | 4 | 0.3022 |
| Trp | 3.9220 | % wt/v | 3.9220 | 204.23 | 11 | 2.5370 |
| Tyr | 0.0055 | % wt/v | 0.0055 | 181.19 | 9 | 0.0033 |
| Val | 0.7056 | % wt/v | 0.7056 | 117.15 | 5 | 0.3617 |
| | | | | | Total | 23.3591 |
| Net Gain* | | | | | Carbon (g) | 0.6191 |

TABLE 2
Sugars, Amino Sugars And Amino Acids With Nitrogen Content Indicated
Original Solution: 100 mL Sucrose in H2O @ 54% M/V
Total grams elemental Carbon in 54 grams C12H22O11:

| Final Product | | | | | | |
|---|---|---|---|---|---|---|
| Analyte | Result | Unit | Total g | Molecular Mass | N per Molecule | Nitrogen (g) |
| Mannose | 1.973 | % wt/v | 1.973 | 180.16 | 0 | 0 |
| Xylose | 5.0472 | % wt/v | 5.0472 | 150.13 | 0 | 0 |
| Arabinose | 1.2444 | % wt/v | 1.2444 | 150.13 | 0 | 0 |
| Galactose | 4.0199 | % wt/v | 4.0199 | 180.16 | 0 | 0 |
| Fructose | 0.0005 | % wt/v | 0.0005 | 164.16 | 0 | 0 |
| Glucose | 4.9859 | % wt/v | 4.9859 | 180.16 | 0 | 0 |
| N-Acetylneuraminic acid | 4.7759 | % wt/v | 4.7759 | 309.27 | 1 | 0.2163 |
| N-Acetylgalactosamine | 1.8467 | % wt/v | 1.8467 | 221.21 | 1 | 0.1170 |
| N-Acetylglucosamine | 10.8142 | % wt/v | 10.8142 | 221.21 | 1 | 0.6849 |
| Ala | 0.0055 | % wt/v | 0.0055 | 89.09 | 1 | 0.0009 |
| Arg | 0.0055 | % wt/v | 0.0055 | 174.2 | 4 | 0.0018 |
| Asx | 0.0055 | % wt/v | 0.0055 | 132.12 | 2 | 0.0012 |
| Cys | 0.2844 | % wt/v | 0.2844 | 121.16 | 1 | 0.0329 |
| Glx | 2.3521 | % wt/v | 2.3521 | 146.14 | 2 | 0.4510 |
| Gly | 0.0055 | % wt/v | 0.0055 | 75.07 | 1 | 0.0010 |
| His | 0.0055 | % wt/v | 0.0055 | 155.15 | 3 | 0.0015 |
| Ile | 0.0656 | % wt/v | 0.0656 | 131.17 | 1 | 0.0070 |
| Leu | 7.3243 | % wt/v | 7.3243 | 131.17 | 1 | 0.7823 |
| Lys | 0.0055 | % wt/v | 0.0055 | 146.19 | 2 | 0.0011 |
| Met | 0.0055 | % wt/v | 0.0055 | 149.21 | 1 | 0.0005 |
| Phe | 0.5853 | % wt/v | 0.5853 | 165.19 | 1 | 0.0496 |
| Pro | 0.0055 | % wt/v | 0.0055 | 115.13 | 1 | 0.0007 |
| Sar | 0.0055 | % wt/v | 0.0055 | 105.09 | 1 | 0.0007 |
| Thr | 0.7494 | % wt/v | 0.7494 | 119.12 | 1 | 0.0881 |
| Trp | 3.9220 | % wt/v | 3.9220 | 204.23 | 2 | 0.5381 |
| Tyr | 0.0055 | % wt/v | 0.0055 | 181.19 | 2 | 0.0004 |
| Val | 0.7056 | % wt/v | 0.7056 | 117.15 | 1 | 0.0844 |
| | | | | | | 3.0613 |
| Net Gain* | | | | | Nitrogen (g) | 3.0613 |

TABLE 3
Total Amino Acid/Protein, Sugar and Carbohydrate Analysis

| Analyte | Result | Unit |
|---|---|---|
| Sugars Carbohydrates | 34.71 | % wt |
| Amino Acids/Protein | 15.99 | % wt |
| Moisture | 46.71 | % wt |
| Ash (inorganics) | 1.1 | % wt |
| Fat (hexane soluble) | 0.3 | % wt |
| SUM | 98.81 | % wt |

The end products indicated in Tables 1-3 confirm that fossilized organic matter (FOM) has catalyzed glycolysis in a saccharide reaction, as indicated by the decomposition of sucrose and the rearrangement of its monosaccharides. Nitrogen fixation and amino acid/protein formation is confirmed by the increased nitrogen in the reaction products as compared to the starting materials, as well as the numerous amino acids produced. Glycosylation is confirmed by the formation of the three acetyl amine compounds. Carbon fixation is indicated by the increase in carbon content of the end products as compared to the sucrose starting material. While the FOM itself included some amino acids and saccharides of its own, the quantities available in one milliliter of a 30% solution do not explain the quantities obtained from one liter of a 54% solution of sucrose.

### CONCLUSION

Thus in the preferred embodiments of the invention, various important aspects include without limitation FOM catalyst, and the use of fossilized organic matter (FOM) as a catalyst for reactions including but not limited to nitrogen fixation, glycosylation, amino acid/protein synthesis, glycolysis, carbon fixation.

Of course it is understood that the foregoing are preferred embodiments of the invention and that various changes and alterations can be made without departing from the spirit and broader aspects of the invention.

1. A method of catalyzing a reaction selected from the group consisting of nitrogen fixation, glycosylation, amino acid/protein synthesis, and carbon fixation, by incorporating a catalyzing amount of fossilized organic matter into the reaction mixture.

2. The method of claim 1, in which the reaction catalyzed is nitrogen fixation, in which nitrogen is reacted with the carbohydrates to form amino acids.

3. The method of claim 1, in which the reaction catalyzed is the polymerization of the amino acids to form proteins.

4. The method of claim 1, in which the reaction catalyzed is glycosylation, in which a sugar molecule is added to the nitrogen atom in a protein or amino acid.

5. The method of claim 1, in which the reaction catalyzed is carbon fixation, in which carbon dioxide is incorporated into carbohydrates.

6. The method of claim 1, in which the fossilized organic matter is used to catalyze multiple reactions with saccharides, including: nitrogen fixation to form amino acids, and glycosylation to form acetyl amine compounds.

7. The method of claim 6 in which said multiple reactions include carbon fixation.

8. The method of claim 1 in which the reaction is conducted in aqueous solutions, suspensions, slurries and any combination thereof and the ratio of fossilized organic matter to reactant(s) is from about 1:50 to about 1:2500.

9. The method of claim 8 in which said reaction at a pH of 4.5 or less.

10. The method of claim 9 in which said reaction is conducted in a temperature range of from about 15° C. to about 30° C. in a nitrogen containing environment.

11. The method of claim 10 in which light is employed to enhance said reaction.

12. The method of claim 10 in which said fossilized organic matter is reacted as a liquid containing the fossilized organic matter in a concentration of from approximately 10 to about 80%.

13. The method of claim 1 in which said fossilized organic matter is extracted from fossilized soil containing plant organic material in which the cell walls and fibrous material making up plant material has been removed, leaving behind cellular cytosol materials, including crystalline (shales) organic mineral material, amino acids, saccharides, enzymes, cellular salt complexes.

14. The method of claim 1 in which said fossilized organic matter is extracted from fossilized soil containing significant lignite and Leonardite components.

15. The method of claim 1 in which said fossilized organic material is fossilized plant material.

16. The method of claim 15 in which said fossilized plant material constitutes less than 10% by volume of non-plant organic material.

17. The method of claim 1 in which said fossilized organic matter is extracted from fossilized soils using water at a slightly acidic pH of from about 4 to about 7.

18. The method of claim 17 in which said water is mixed with a water-miscible organic co-solvent selected from the group consisting of alcohols, dimethylsulfoxide (DMSO), N,N-dimethylformamide (DMF), acetonitrile, tetrahydrofuran (THF), and p-dioxane.

19. The method of claim 1, in which iron contained in said fossilized organic matter is complexed with phosphate, to form iron (III) phosphate, thereby rendering it insoluble to slightly soluble in water, depending on pH, and in which said complexed iron is removed from said fossilized organic matter prior to using it in said reaction.

20. A method for converting sucrose at least in part to other ingredients by incorporating into a solution of sucrose at a Brix level between about 20 and 80, an amount of fossilized organic matter in a ratio of fossilized organic matter to sucrose of from about 1:50 to about 1:2500, at a pH of 4.5 or less, and at a temperature of from about 15° C. to about 30° C. in a nitrogen containing environment, and in which said fossilized organic matter is reacted as a liquid containing the fossilized organic matter in a concentration of from approximately 10 to about 80%.

21. The method of claim 1, in which said fossilized organic matter is impregnated onto a high surface area solid catalyst support placed in a reactor though which reactants are continuously fed.

22. The method of claim 21 in which said impregnation step is conducted by soaking said solid support in a liquid extract solution of fossilized organic matter at a concentration of from approximately 10 to about 80%.
